(19) United States
(12) Patent Application Publication
(10) Pub. No.: US 2026/0151030 A1
(43) Pub. Date: Jun. 4, 2026

(54) SCANNING LASER OPHTHALMOSCOPE FOR SMALL ANIMALS

(71) Applicant: VOX IMAGING TECHNOLOGY LTD, London (GB)

(72) Inventors: Peter West, Deal (GB); Alan Robinson, Harlow (GB)

(73) Assignee: VOX IMAGING TECHNOLOGY LTD, London (GB)

(21) Appl. No.: 19/123,972

(22) PCT Filed: Oct. 23, 2023
(86) PCT No.: PCT/GB2023/052777

§ 371 Date: Apr. 24, 2025

Related U.S. Application Data
(60) Provisional application No. 63/380,605, filed on Oct. 24, 2022.

(51) Int. Cl. A61B 3/10; A61B 3/12

(52) U.S. Cl. CPC A61B 3/102; A61B 3/1025; A61B 3/1241

(57) ABSTRACT

A compact confocal scanning laser ophthalmoscope comprising a MEMS scan mirror with a compact scan head, for use with small animal subjects. The scan head is connected to the source, detector and control electronics by flexible optical and electrical umbilicals and includes a zoom lens to provide both a wide field of view and high resolution imaging of the retina using a range of imaging modalities.

### FIELD OF THE INVENTION

This invention relates to a confocal scanning laser ophthalmoscope for examining the fundus, that is the inside back surface of the eye, of small animals.

### BACKGROUND OF THE INVENTION

There is increasing use of fundus imaging of small animals, in basic research, in drug discovery and in pre-clinical investigations of treatments for ophthalmic disease. Typical subjects include mice, other rodents, and zebra fish. These animals are used to study the physiology of vision, as models for the development of human eye disease, and in the development of treatments for a wide range of diseases of the eye and the nervous system.

Existing instruments include conventional fundus cameras. These are well known, and are described, for example by Sato in U.S. Pat. No. 3,016,000 and more recently, specifically targeting small animals, by Massie in U.S. Pat. No. 7,993,000. A problem with such direct imaging instruments is the need to simultaneously illuminate the entire field of view on the retina. Scattering of light in the lens and vitreous humour of the subject's eye causes undesirable loss of contrast.

Pomerantzeff and Webb in U.S. Pat. No. 4,213,678 describe a scanning ophthalmoscope which mitigates these problems by scanning a laser spot focused onto the retina, so that at any instant only a small fraction of the field of view is illuminated. A further enhancement is described by Webb, Hughes and Delori (1987), who insert a pinhole spatial filter in the detection path which is confocal with the small area of the retina illuminated by the scanned laser and suppresses light scattered from other surfaces. Another example of such a confocal scanning instrument is described by Blaha and Gaida in U.S. Pat. No. 5,071,246.

Scanning laser opthalmoscopes have been used for fundus imaging of small animals. Unfortunately, clinical instruments designed for human subjects are bulky and poorly adapted for use with smaller subjects. Research instruments, such as those described by Zhang et. al. (2015) are similarly inconvenient to use. There is a need for an instrument whose ergonomics are suited to research on small animal subjects.

The present invention combines the small size and ease of use associated with small fundus cameras, with improved optical contrast, high lateral and axial optical resolution, and a wide range of imaging modalities made possible by a confocal scanning mechanism.

A distinguishing feature of a confocal scanning laser ophthalmoscope (cSLO) is the scan mechanism, which deflects both the incident laser beam, and the scattered or fluorescence signal returned from the subject. Ideally, the incident and returned beams are substantially coaxial and tilt in unison about a point within the eye, close to the centre of the pupil or to the anterior nodal point of the eye. This is typically achieved using one or more rotating, tilting or vibrating mirrors to execute a two-dimensional raster scan. An optical relay is provided such that the mirror tilt axes are at conjugate foci with respect to the beam tilt axes in the subject's eye.

The detected signal is maximised when the effective diameter of the beam returned to the detector is constrained only by the subject pupil diameter. As discussed by Webb et. al. (1987), in the absence of vignetting and for a collimated beam, the product of beam area and the solid angle swept out by the scan is an optical invariant of the system between pupil and scan mirror. The beam width and deflection range at the subject's pupil determines the minimum size and deflection capability required for the mirror. In terms of the mirror properties, the product of beam area at the scan mirror and the sines of the orthogonal mirror deflection angles is substantially independent of the magnification of the optical relay. It follows that for a given beam diameter and deflection angle at the scan mirror, an increase in magnification at the pupil results in a smaller deflection angle at the pupil.

The dilated pupil diameter of a human eye can be as large as 8 mm, with an effective focal length approximately 17 mm. A mouse eye is smaller, with a 2 mm pupil and effective focal length typically around 1.9 mm. For the same deflection angle, a much smaller scan mirror can be used for small animal subjects.

Webb (1987) describes a scan system with a polygon mirror rotating at 37800 rpm, comprising 25 6 mm facets which are over-filled by the return beam from a dilated 8 mm diameter human pupil. He also describes another unit magnification configuration with a 5 mm diameter galvanometer mirror driven at resonance, which would similarly fail to capture all the light returned from the pupil. For the slower vertical scan, a larger galvanometer mirror located at a separate pupil conjugate is employed.

A high frame rate is desirable. Animal subjects are typically anaesthetised during ophthalmic examinations, but this does not suppress all movements of the eye, notably those related to beating of the subject's heart. To reduce motion blur individual images should be captured within a sufficiently short time period to minimise any loss of resolution from such movement. Frame rates less than 15 Hz are problematic, and 30 Hz or greater is preferred.

To sample 500 lines across the field of view using a simple raster scan at 30 Hz, a line rate of 15000 Hz is required. A rotating polygon mirror can achieve this while maintaining a constant deflection rate for the beam in one direction. A second deflecting mirror is required to scan in the orthogonal direction, ideally located at an optical conjugate of the polygon facet. A mirror driven by a galvanometer near its resonant frequency is an alternative, but in existing instruments a second mirror is required for a two-dimensional scan at line rates greater than 10000 Hz. Instruments built along these lines are bulky and inconvenient to use with small animals.

A simpler and more compact optical system is possible if a single mirror deflects the beam in two orthogonal directions. The present invention recognizes that for the small diameter of a mouse pupil, two-dimensional scanning using such a single reflecting surface is a practical approach. In particular, MEMS (micro electro-mechanical system) technology is used to manufacture such mirrors for optical projectors, typically employing lithographic patterning and etching of silicon, as described for example in US2010/0020379. Devices of this type can achieve combinations of field of view, beam width at the subject pupil, scan rate and optical resolution appropriate for a small animal SLO. A further advantage of such a MEMS mirror is that power consumption is much lower than the several watts which is common for galvanometer scanners.

MEMS resonant scanners designed to operate at frequencies of 7.5 kHz or higher typically have mirror diameters around 1 mm or less and can deflect the scan beam through angles of 20° to 45°. With suitable relay optics a wider deflection angle is possible at the pupil, but this is at the expense of reduced beam width, which in turn increases the size of the retinal spot through diffraction. The loss of resolution is not a concern for wide field imaging, given that a frame rate of 30 Hz or higher limits the number of lines that can be scanned within a single frame. In this regime, a scan spot whose width increases in proportion to the field of view is beneficial in avoiding under-sampling the subject and reduces artefacts due to aliasing.

There is a conflict between the requirements for a wide field of view needed for navigation and identification of retinal features of interest, in contrast to high resolution imaging for more detailed study of the areas of interest. In existing instruments, this is resolved by providing multiple sets of relay optics to image the scan mirror into the subject pupil, each with different magnification. A change of magnification requires that one optical relay unit is removed and replaced by another. A more convenient arrangement is to change the magnification of the relay optics by an internal mechanism, while maintaining a fixed distance between the final element of the tube lens and the subject, constraining the tilt axes of the beam close to the subject pupil, and keeping the scan spot focused at the retina.

Gray (2017) in U.S. Pat. No. 9,743,831 suggests the use of a MEMS scan mirror with reflective optics and without confocal spatial filtering. Gray notes that such a 2-D scanning device should provide a scan deflection of 180 degrees or more, which is not possible with readily available silicon MEMS components meeting other essential SLO requirements including mirror diameter and deflection rate or resonant frequency. A more practicable scheme proposed by Gray comprises a one-dimensional scan mirror mounted on a co-axial rotating shaft, which can achieve large deflection angles in at least one direction, but this will be bulkier than relying on MEMS technology for both deflection axes.

Another aspect neglected in Gray's U.S. Pat. No. 9,743,831 disclosure is vergence, or degree of convergence or divergence of the scanned beam at the subject pupil. An ability to control vergence at the pupil is essential to focus the scanned beam on the parts of the retina to be imaged. This is addressed for human subjects by Blaha and Gaida (U.S. Pat. No. 5,071,246, 1991), but is of particular importance for small animal subjects in which the relatively large ratio of pupil diameter to focal length limits the axial defocus which can be tolerated. A confocal instrument, as disclosed in this application, exploits the limited depth of focus to distinguish image structures at disparate depths within the retina, while suppressing signals from adjacent anterior or posterior layers.

The ocular refractive error of small rodents varies significantly, not only between individual subjects, but also with the age of individual specimens as reported by Zhou (2008), and with wavelength and location across the retina as reported by Geng (2011). To this natural variation of approximately 70 Dioptre, an additional 50 Dioptre of adjustment is needed to adjust focus from the anterior neural fibre layer to the posterior retinal pigment epithelium, as reported by Lee et. al (2013).

For small animal imaging, incorporating the scan optics into a physically compact scan head greatly facilitates physically rotating the entire scan head about the subject pupil to position a region of interest of the retina near the centre of the field of view, rather than presenting the animal to a bulky static instrument designed for human subjects.

Support for multiple imaging modes is necessary for many applications. Modalities in use include reflectance imaging at visible and near infra-red wavelengths, fundus fluorescence imaging at one or more wavelengths, and autofluorescence at visible or infra-red wavelengths. Operational flexibility without compromise to the compact form and ergonomics of the scan head is provided by locating components including illumination sources, wavelength multiplexers, filters to separate excitation and fluorescence signals (wavelength demultiplexing), detection and control electronics in a separate unit, connected to the scan head by flexible electrical and optical fibre umbilicals. An optical umbilical is particularly advantageous for techniques such as optical coherence tomography (OCT) or fluorescence lifetime spectroscopy where the detection equipment or illumination source can be substantially larger than a simple semiconductor detector or laser diode.

While confocal imaging can discriminate between different layers in the retina, axial resolution is limited by the numerical aperture (NA) at the retina, approximately equal to the beam radius at the pupil divided by the effective focal length of the subject's eye. For small beam radii, the axial resolution is inversely proportional to the square of the NA. For wider beams, the theoretical resolution is further degraded by optical aberrations in the subject's eye. Optical coherence tomography (OCT) offers a useful improvement in axial resolution which is independent of the beam width, as explained by Tomlins and Wang (2005). OCT is increasingly used in ophthalmic imaging.

OCT employs a broad-spectrum source with low temporal coherence, and mixes light reflected from the subject with light from a reference optical path. Strong coherent interference between the two beams is observed only when their optical path lengths differ by less than the coherence length of the source. Ophthalmic OCT typically employs a depth first scanning mode, in which an axial scan (A-scan) is recorded sequentially for each point of interest along a scan line to create a 2-dimensional B-scan. A volume scan is captured by recording a series of parallel B-scan slices. A-scan rates are relatively slow, with repetition rates typically less than 100 kHz, and incompatible with the high-speed resonant scan mirror required for this invention.

Podoleanu and Jackson in U.S. Pat. No. 5,975,697 describe an en-face imaging system in which a phase or frequency modulation is applied to the reference optical path so that coherent interference and detection results in an oscillating electrical signal. Band-pass filtering and demodulation produce a signal indicative of the optical power reflected from the subject at an axial depth corresponding to the current reference path length. This requires relatively high-speed detection electronics compared with more conventional SLO operation but allows a 2-dimensional en-face scan to be recorded at the same frame rate as an SLO scan with the same lateral resolution. Depth information is recovered by changing the length of the reference optical path, typically by moving a reference reflector, and hence capturing successive frames at different depths within the subject's eye.

An advantage of en-face scanning is that retinal focus can be adjusted to match the depth of the en-face scan, using a beam width at the pupil which optimises lateral resolution. This reduces the axial depth of focus. In contrast, smaller beam widths with poorer lateral resolution are preferred with conventional depth-first A-scan approaches, especially when using Fourier domain or swept spectrum OCT.

The need for high-speed modulation and detection is avoided if the reference signal is split into two parallel paths, whose lengths differ by one quarter of the centre wavelength of the source. Light reflected from the subject is also split and mixed with the reference beams in two differential interferometers, such that their outputs represent quadrature components of the beat signal. This technique is widely employed in coherent optical communications systems at high gigahertz frequencies, but has also been used for optical coherence elastography at audio frequencies by Adie et. al. (2009).

Tissue is typically birefringent, causing the polarization state of light to evolve during propagation. In conventional OCT, only the component of the returned signal in the same polarization state as the reference contributes to the detected signal, reducing the image intensity from parts of the subject. Polarisation fading can be avoided by detecting the signal returned in each of two orthogonal polarization states and summing the contributions. Commercial modules such as Thorlabs INT-POL-1300 provide this capability.

Polarisation sensitive optical coherence tomography exploits tissue birefringence to image tissue structures, as described by de Boer (2017). By applying the quadrature detection scheme described above to both orthogonal polarization components of the returned signal, the intensity and polarization state of the return signal can be determined from four simultaneous measurements of the in-phase and quadrature field in the two polarization states. This both eliminates polarization fading and enables en-face polarization sensitive OCT of the subject.

### SUMMARY OF THE INVENTION

This invention discloses a scanning laser ophthalmoscope to examine the fundus or retina of small animals. The scan head incorporates a scan mirror manufactured as a micro electro-mechanical system (MEMS) component, with means to tilt about two orthogonal axes, and typically etched from silicon. In a preferred embodiment, one axis of the scan mirror is driven to excite a mechanical resonance such that a wide deflection angle is achieved with low power consumption.

Use of a single scan mirror simplifies the optical design. Means are provided for rapid adjustment of the axial focus of the instrument, an optical relay for efficient coupling of the scanned beam into the subject's eye and for collection and confocal spatial filtering of light returned from the fundus. Preferably, the optical relay allows the user to zoom or vary the field of view at the subject so that regions of interest can be imaged at higher magnification with enhanced optical resolution and signal to noise ratio. Axial sectioning to extract 3-dimensional information is possible using a rapid focus capability in concert with confocal spatial filtering. These means are combined into a compact unit providing greatly improved performance and ergonomics compared with existing instruments for small animal investigations.

In preferred embodiments a flexible optical interconnect such as an optical fibre cable transfers reflected or fluorescence emission from the subject to a detection system. Operational flexibility is enhanced if excitation illumination is delivered via a flexible optical waveguide carrying a single spatial mode from one or a multiplicity of selectable sources. The axial resolution and sectioning capability can be enhanced by an optical coherence tomography module employing quadrature detection to support en-face imaging at similar frame rates and field of view as other imaging modes.

According to one aspect of the present disclosure, there is provided an ophthalmic imaging apparatus comprising an optical coherence tomography device. The optical coherence tomography device comprises: an optical source with low temporal coherence; an optical splitter to direct the output from said optical source simultaneously to a subject and to a reference optical path comprising a delay module to vary the optical path length of said reference path optical path; and an optical combiner to combine light reflected or scattered from said subject with light transmitted through the reference optical path, wherein in-phase and quadrature interference beat signals are generated by a quadrature detector comprising a 90-degree optical hybrid and balanced optical detection.

According to another aspect of the present disclosure, there is provided an ophthalmic imaging means comprising an optical coherence tomography means further comprising: an optical source with low temporal coherence; means to direct output from said optical source simultaneously to a subject and to a reference optical path comprising a means to vary the optical path length of said reference path optical path; means to combine light reflected or scattered from said subject with light transmitted through the reference optical path wherein in-phase and quadrature interference beat signals are generated by a quadrature detector comprising a 90-degree optical hybrid and balanced optical detection.

According to yet another aspect of the present disclosure, there is provided a method of imaging the fundus of a small animal (for example a zebra fish or a rodent, for example a mouse), comprising using the ophthalmic imaging apparatus according to the first, or other, aspects of the present disclosure.

According to a further aspect of the present disclosure, there is provided an instrument comprising an optical scan head, said scan head comprising: an optical excitation light source with high spatial coherence; a first optical system to direct light from said optical excitation source onto a scan mirror; a variable focusing optic to vary the vergence of the excitation light directed by said first optical system onto said scan mirror; a second optical system which forms an image of the scan mirror near the pupil of the eye of a subject such that the excitation light is brought to a focus in or near the retina of the subject's eye; a beam splitter to separate light returned from said retinal focus point from the path of the excitation beam after said returned light is reflected by the scan mirror, and a lens to focus it to a point conjugate with the retina; a spatial filter located at said retinal conjugate; and a flexible coupler to transfer light transmitted by said spatial filter to a detection system. The scan mirror may be part of a micro electro-mechanical system comprising: said scan mirror able to tilt about a first axis substantially aligned with a first diameter of said mirror; and a driver to excite a resonant oscillation of said mirror about said first axis and an actuator to tilt said mirror about a second axis substantially aligned with a second diameter of the mirror and orthogonal to the first axis.

According to a further aspect of the present disclosure, there is provided an instrument comprising an optical scan head, said scan head comprising: an optical excitation light source with high spatial coherence; a first optical system to direct light from said optical excitation source onto a scan means comprising a scan mirror; a focus means to vary the vergence of the excitation light directed by said first optical system onto said scan mirror; a second optical system which forms an image of the scan mirror near the pupil of the eye of a subject such that the excitation light is brought to a focus in or near the retina of the subject's eye; a means to separate light returned from said retinal focus point from the path of the excitation beam after said returned light is reflected by the scan mirror and to focus it to a point conjugate with the retina; a spatial filter means located at said retinal conjugate; a flexible coupling means to transfer light transmitted by said spatial filter to a detection means; wherein said scan mirror is part of a micro electro-mechanical system comprising: said scan mirror able to tilt about a first axis substantially aligned with a first diameter of said mirror; a means to excite a resonant oscillation of said mirror about said first axis; a means to tilt said mirror about a second axis substantially aligned with a second diameter of the mirror and orthogonal to the first axis.

It will be appreciated that features described in relation to one aspect of the present invention may be incorporated into other aspects of the present invention. For example, the apparatus of the first aspect of the invention may incorporate any of the features described with reference to apparatus of further aspects of the invention and vice versa.

### BRIEF DESCRIPTION OF DRAWINGS

FIG. 1 is a schematic of the optical configuration, signal processing and control system.

FIG. 2 shows a collimated beam projected through the pupil of the subject's eye, focused at the retina, and scattered light exiting the pupil substantially parallel to the incident beam.

FIG. 3 illustrates a tilted beam entering the pupil with light scattered from the retinal spot exiting the pupil substantially parallel to the incident illumination.

FIG. 4 shows how the vergence of light incident at the pupil determines the depth within the retina at which the light is brought to a focus.

FIG. 5 shows a 4-f optical relay used to image the scan mirror into the subject's pupil.

FIG. 6 shows a variable magnification optical relay in a low magnification configuration with wide angular deflection of the beam at the subject.

FIG. 7 shows a variable magnification optical relay in a high magnification configuration with reduced angular deflection of the beam at the subject.

FIG. 8 illustrates an embodiment of the detection system with two optical detectors responding to different wavelengths selected by re-configurable optical filters.

FIG. 9 is a schematic of an optical zero crossing detector used to synchronise the timing of signal sampling and digitisation to the scan mirror deflection.

FIG. 10 illustrates the internal configuration of a compact scan head achieved using an optical path folded around a robust chassis.

FIG. 11 shows a control unit and a table providing stable and adjustable mounting for both the scan head and a cradle to support a small animal subject.

FIG. 12 shows an excitation source able to provide illumination at multiple wavelengths with provision for optical fibre coupled optical coherence tomography.

FIG. 13 illustrates an implementation of optical coherent tomography supporting en-face imaging using phase modulation of the reference path and band-pass filtering and demodulation of the electrical beat signal.

FIG. 14 shows an alternative OCT reference path and demodulation means employing acousto-optic modulation, balanced detection and phase sensitive recovery of the coherence beat signal.

FIG. 15 shows an implementation of en-face OCT using a 90-degree optical hybrid and two balanced detectors to recover in-phase and quadrature beat components.

FIG. 16 shows a preferred implementation of en-face OCT using a 90-degree optical hybrid in which the OCT probe is coupled to the scan head using a dedicated fibre coupling port.

FIG. 17 shows a polarization diverse OCT detection scheme using a 90-degree optical hybrid with balanced detectors for two orthogonal polarization states of the signal.

### DETAILED DESCRIPTION OF THE INVENTION

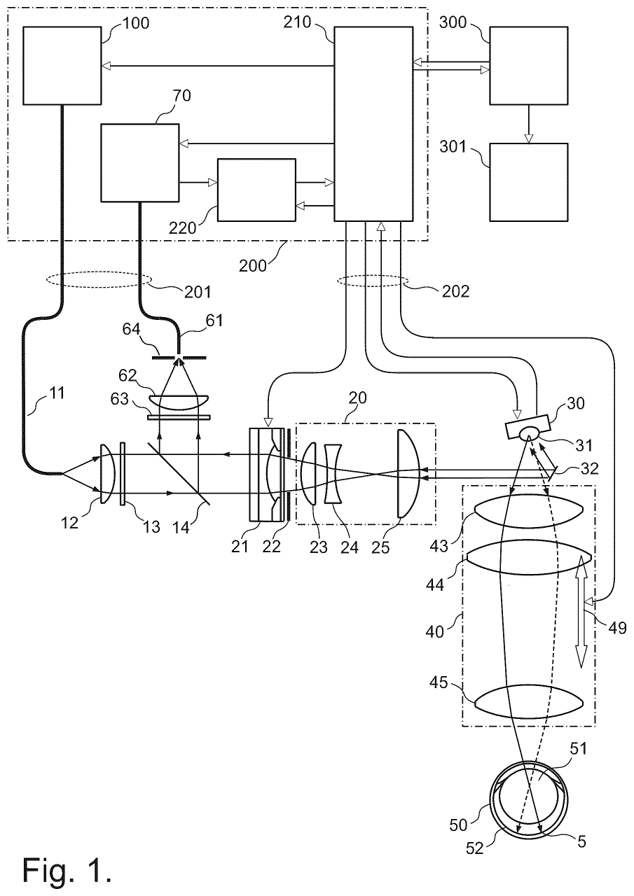

The invention will be described, first in outline with reference to FIG. 1, and subsequently in more detail. Light from excitation source 100 is guided by flexible waveguide 11 to a fibre coupling assembly whose lens 12 directs a substantially collimated beam of light through beam splitter 14 into focusing means 21 and first optical relay system 20, whose lenses 23, 24, 25, project an image of aperture 22 onto a scan mirror 31, driven by the MEMS module 30, after reflection at the turning mirror 32.

A second optical relay system 40 projects an image of the scan mirror into the pupil 51 of the subject's eye 50. Light emitted from the scan spot 5 and exiting the pupil is focused back onto the scan mirror 31, and re-traces the path of the excitation beam as far as the beam splitter 14 where it is deflected into a fibre coupler whose lens 62 focuses the returned light onto an aperture 64. The aperture may comprise the core of optical fibre waveguide 61 which transmits the captured light to detection system 70.

The scan mirror and associated optics are contained within a compact scan head, connected by optical umbilicals 201 comprising optical fibres 11, 61, and by electrical umbilical 202, to a base unit comprising the excitation source 100, detection system 70, signal processing module 220 and control module 210.

The detector output is digitised by signal processing module 220 and streamed to the control module 210 which communicates with host computer 300 which in turn forwards user commands to the instrument and displays images from the instrument on screen 301. The control module 210 sends configuration commands to instrument sub-systems including, but not limited to the excitation source 100, focus means 21, the driver to the MEMS module 30, zoom magnification actuator 49 for the second optical relay 40, and optical filter selection in the detection system 70.

The invention will now be described in more detail. A scanning laser ophthalmoscope (SLO) projects a substantially collimated beam of light into the subject's eye, where it is brought to a focus at the retina. Light scattered, reflected or generated by fluorescence is emitted from the small, illuminated volume, and exits the subject pupil. A fraction of this light is intercepted and measured by the instrument. A distinguishing feature of an SLO is that light is returned predominantly only from those parts of the eye illuminated by the scanned beam, which improves image contrast compared with a traditional fundus camera which flood-illuminates the retina and collects light simultaneously from all parts of the eye.

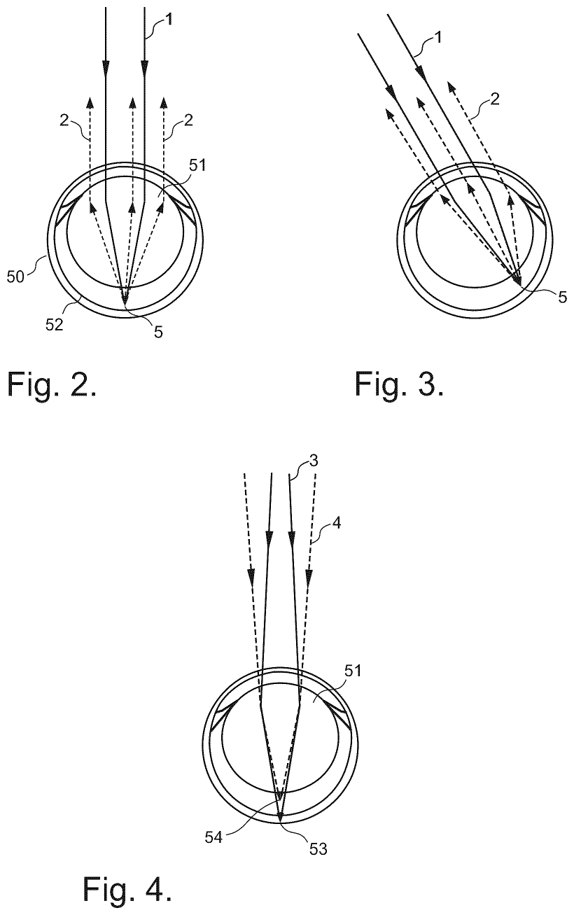

FIG. 2 shows a collimated beam 1 incident through the pupil 51 of a subject's eye 50 and focused onto a small spot 5 on the retina 52. Light 2 emitted from spot 5 and exiting the pupil is substantially collimated and emerges parallel to the incident beam. FIG. 3 shows that as the incident beam 1 is tilted by the illumination system, the spot 5 is brought to focus at a different point on the retina, but the returned light 2 remains parallel to the incident illumination, and so can be intercepted by the scan mirror.

A further substantial improvement in contrast is achieved by confocal spatial filtering of the returned beam. The spatial filter shown in FIG. 1 can be a physical aperture 64, or the core or mode field of an optical waveguide 61, and is arranged to be confocal with the scanned spot on the retina. In the absence of scattering along the optical path, only light emitted from or very close to the scan spot reaches the detector. In practice, some scattering is unavoidable, but the proportion of scattered light reaching the detector decreases rapidly with increased separation between the scattering site and the point of focus on the retina.

Selective collection of light emitted close to the plane of focus makes it possible to generate images of structures at different depths within the retina. A means to vary the focus is necessary to exploit this, to accommodate variations of refractive error between subjects, and in individual subjects to accommodate variations with age, with position across the retina, and for different wavelengths of light.

FIG. 4 shows the effect of changing the vergence of the scan beam at the pupil 51. Vergence is a measure of how strongly the illumination converges towards or diverges from the surface of interest, and is quantified by the reciprocal of the distance from the point of divergence or convergence to said surface. For diverging beam 3 vergence is negative, and the point of focus 53 is displaced towards the back of the eye. For converging beam 4 the point of focus 54 is displaced towards the front of the eye. Preferably the incident beam remains centred in the pupil 51, and the width at the pupil is substantially unchanged as the vergence is varied.

The retinal thickness of a mouse eye is typically around 0.2 mm, the effective focal length 1.9 mm, and the refractive index of the vitreous medium 1.34. A variance range of approximately 50 Dioptre (50 m⁻¹) at the pupil is needed to vary focus through the thickness of the retina, and 70 D to accommodate variations across the retina and between subjects, for a total vergence range of 120 Dioptre.

Focusing can be implemented using conventional glass or polymer optics, with means to adjust the position of one or more elements. Ideally the response time should be comparable with or less than the period of a single video frame, typically 30 ms, so that a rapid sequence of images can be captured at different axial depths within the retina. In a preferred embodiment, a lens element with variable focal length is used. This may comprise an electro-wetting lens, such as a Varioptic lens of the type manufactured by Corning inc., a shape-changing lens such as that manufactured by Optotune, which employs electromagnetic actuation to change the curvature of a transparent membrane via hydraulic pressure, or another component with equivalent functionality.

The instrument described by Blaha (U.S. Pat. No. 5,071,246) has two independent focus adjustments for incident and reflected light respectively. In the instrument shown in FIG. 1, the focusing element is common to both excitation and detected light paths, with the returned signal separated by a beam splitter 14 after transmission through the focus means 21. This ensures that in reflectance imaging, changes in axial focus for the beam incident on the subject produce an identical change in the returned signal, so that the contra-propagating beams have identical vergence at the beam splitter.

The spatial resolution of the instrument is determined predominantly by the size of the spot which is focused onto the retina. A small spot size is most easily achieved if the light is spatially coherent across the emitting region. In a preferred embodiment the excitation source is delivered from an optical waveguide which supports a single transverse optical mode, such as a single mode optical fibre.

Semiconductor lasers and super-luminescent light emitting diodes (SLED) are convenient sources in which the active region is an optical waveguide so that the mode field has high spatial coherence and can be coupled into a polarisation-maintaining optical fibre with high efficiency. More broadly, fibre coupling allows bulkier super-continuum sources or mode locked lasers to be used without compromising the ergonomic benefits of a compact scan head.

It is advantageous to support multiple excitation wavelengths and provide a capability to employ different imaging modalities simultaneously. For example, fluorescence imaging excited by 480 nm or shorter blue wavelengths combined with reflectance imaging at a near infra-red wavelength such as 785 nm.

Multiple laser sources can be combined using lenses to collimate their outputs, dichroic filters to combine the collimated outputs and a final lens to couple the multi-wavelength beam into the excitation fibre. Fused tapered fibre couplers are a viable alternative, capable of low insertion loss and avoid the need for careful alignment of the merged optical beams. With suitable equipment, fusion splicing of pigtailed laser sources to the fibre coupler is straightforward and will usually entail lower insertion loss than fibre connectors relying on precision ferrules for alignment.

A concern when excitation sources extend over a broad wavelength range is ensuring single mode operation at the shortest wavelength while maintaining acceptable bend tolerance of the flexible interconnect at the longest wavelengths. Optical waveguides are characterised by their normalised frequency or V-number, defined by:

$$V = \frac{2 π a}{λ} \sqrt{{n}_{1}^{2} - {n}_{0}^{2}}$$

where λ is the operating wavelength, α is the radius of the core, n₁ is the peak refractive index of the core and n₀ is the refractive index of the cladding. In theory, an optical fibre with cylindrical core and step refractive index profile supports only two polarisations of a single transverse mode for V less than 2.405. In the weakly guided approximation, the linearly polarised fundamental modes are designated LP01. For V-numbers greater than 2.405, additional LP11 modes are supported. This is undesirable, leading to instability in the mode field and the shape and position of the spot focused onto the subject's retina.

For smaller V-numbers, notably those less than 1.5, optical fibres become increasingly sensitive to bends in the waveguide. A significant improvement in bend performance is possible if the refractive index between core and cladding is increased, but this requires a smaller core to maintain single mode operation, resulting in less efficient coupling and degraded tolerance to misalignment in fibre connectors.

In a preferred implementation, the excitation fibre is single mode with normalised frequency greater than 1.5 at the longest illumination wavelength. At the shortest wavelength, the fibre may no longer be strictly single mode. However, the LP11 modes are anti-symmetric with mode field amplitude falling to zero in the centre of the core. Excitation of these LP11 modes is minimised by accurate co-axial alignment between cores when coupling into or between fibres. Excitation of higher order LP02 and LP21 modes is avoided by ensuring that the normalised frequency V is less than the theoretical cut-off value of 3.83, or potentially rather higher for practical refractive index profiles.

Further suppression of higher order modes is possible, for example by locally stretching and thinning the fibre core, so that that the V-number falls below 2.4, and LP11 and higher order modes radiate into the transparent cladding medium. Such a mode filter can be manufactured using apparatus employed for fusion splicing or in the manufacture of fused fibre couplers.

In a preferred implementation illustrated in FIG. 1, the output from the excitation fibre 11 is intercepted by lens 12 and directed as a substantially collimated beam into the focusing means 21. The intensity profile across the beam is approximately Gaussian. To prevent unwanted scattering, especially if the beam width at the scan mirror extends beyond the central specular reflecting area of the mirror, the beam is truncated by an aperture 22, which either comprises part of the focusing means, or is located immediately adjacent to it.

Efficient coupling from the aperture to the scan mirror is achieved by a first optical relay 20 which projects a virtual image of the aperture onto the scan mirror 31. Typically, two or more lenses or lens groups 23, 24, 25, are required to control both magnification and vergence of the image of the aperture at the scan mirror. By locating the aperture and scan mirror at conjugate locations, the size of the beam incident on the scan mirror is independent of changes in beam divergence induced by the focus means. To minimise stray light scattering from the mirror, the magnification of the optical relay is chosen such that the diameter of the virtual image of the aperture is slightly smaller than the central reflective portion of the scan mirror.

It will be appreciated by one skilled in the art that plane (i.e., flat) turning mirrors can be introduced at one or more points along the path from lens 12 to scan mirror 30, folding the optical path, and reducing the overall dimensions of the scan head.

In the absence of optical aberrations in both the optical instrument and in the subject's eye, optical resolution and signal to noise ratio are optimised when the width of the scanned beam at the subject's pupil is as large as possible, consistent with avoiding vignetting whereby the outer parts of the beam are intercepted by the iris, or otherwise fail to reach the retina. In this case, the diameter of the diffraction-limited spot which can be focused onto the retina is inversely proportional to the diameter of the beam at the principal surface of the lens, and contributes to a theoretical limit on the resolution achievable when imaging fine structure in the retina.

In practice optical aberrations in the subject will broaden the retinal spot to an extent which increases rapidly with beam diameter. A typical mouse pupil has a diameter of 2 mm. Zhang (2015) found that lateral resolution is optimised for a beam width of 1.3 mm, in broad agreement with simulations based on Zernike aberration coefficients reported in an earlier publication by Geng (2011). For narrower beams, optical diffraction broadens the spot. With wider beams, resolution is degraded by increasing optical aberrations.

Compared with non-resonant devices, a resonant scan mirror increases the rate at which lines are scanned, but limits the size of mirror for acceptable vertical resolution and frame rate. For 320 lines vertical resolution at 30 frames per second a resonant frequency of at least 5 kHz is desirable. 10 kHz or higher is preferable for a higher vertical resolution of 620 lines at 30 Hz or 720 lines at 25 Hz. MEMS mirrors with this performance have been developed for projection applications. Examples include modules with diagonal field of view 50°, and mirror diameter 1 mm.

Scan mirrors with resonant deflection in both axes are available, and trace out a Lissajous scan pattern, rather than a regular raster. For a given frame rate and line count, a uniform linear or staircase ramp in the vertical direction results in slower peak scan velocity, longer integration time and a better signal to noise ratio in the centre of the field of view than a sinusoidal deflection, so are preferred. US 2010/0020379 describes a hybrid scan mirror with a resonant electrostatically driven horizontal axis and an electromagnetically driven vertical axis, in a gimbal configuration such that the rotation axes are coplanar. Resonant oscillation of the inner gimbal provides a high line rate, and the electromagnetic drive of the outer gimbal can execute an accurately stepped staircase deflection to maintain a uniform line spacing throughout the vertical deflection range.

We require that light incident on the scan mirror is projected into the subject's eye and onto the retina, and that a substantial fraction of the light returned from the subject is captured by the detection system. In a preferred embodiment this is achieved by a second optical relay system 40 which projects a virtual image of the scan mirror into the pupil of the subject's eye, such that the scan mirror and the subject pupil are at conjugate locations.

With a well-corrected optical system, the product of solid angle swept out by the mirror and the area of the beam cross-section measured normal to the direction of propagation is constant. Ignoring ocular aberrations, optical resolution is maximised when the beam fills the pupil. However, magnifying the image of the scan mirror at the pupil reduces the sine of the angular deflection of the scan beam by a factor equal to the pupil magnification. The field of view can be increased using a lower transverse magnification. This results in a smaller beam width at the subject pupil. In this regime the lower resolution and larger diffraction-limited spot size at the retina reduces aliasing artefacts which would otherwise arise if the angular separation of successive lines exceeded the optical resolution.

The results from Zhang (2015) show that for a typical mouse subject, ocular aberrations are a limiting factor. Transverse optical resolution is optimised for a beam width at the subject pupil of order 1.3 mm. Independent simulations using aberration coefficients published by Geng (2011) indicate that confocal spatial filtered signal power peaks for a beam width between 0.6 and 1.6 mm. The precise optimum depends on the size of the confocal spatial filter, the intensity profile across the incident illumination beam, and the magnitude and character of the optical aberrations in the subject. In general, a relatively large confocal spatial filter combined with a large beam diameter maximises capture efficiency when the fraction of light returned is low. Higher transverse resolution is possible with tight spatial filtering, such that the conjugate image of the spatial filter is comparable with the diameter of the diffraction-limited Airy disk. Larger beam diameters can improve axial resolution, but there is little benefit from beam diameters larger than 1.6 mm.

For the expected range of ocular aberrations, a beam width of 1.0 to 1.4 mm enables good resolution, combined with good axial resolution as the focus is varied. Using current MEMS technology, 2-axis mirrors with resonant frequency as high as 10 kHz are limited to relatively small diameters of order 1 mm, with diagonal field of view typically 50° or less. Rotation of the mirror from normal incidence reduces its effective diameter, measured in a plane orthogonal to the beam direction. Moreover, to avoid the insertion loss introduced by a beam-splitter, a preferred embodiment employs an off-axis turning mirror 32 which increases the worst-case angle of incidence, and further reduces the maximum beam width at the scan mirror. For angles of incidence at the mirror smaller than 25°, the beam width can be 90% of the mirror diameter, provided it is accurately centred on the rotation axes.

A seamless transition, from a wide field of view for navigation and initial assessment, to a narrower region of interest with high transverse and axial resolution, becomes possible if the magnification of the optical relay 40 can be controlled by the user.

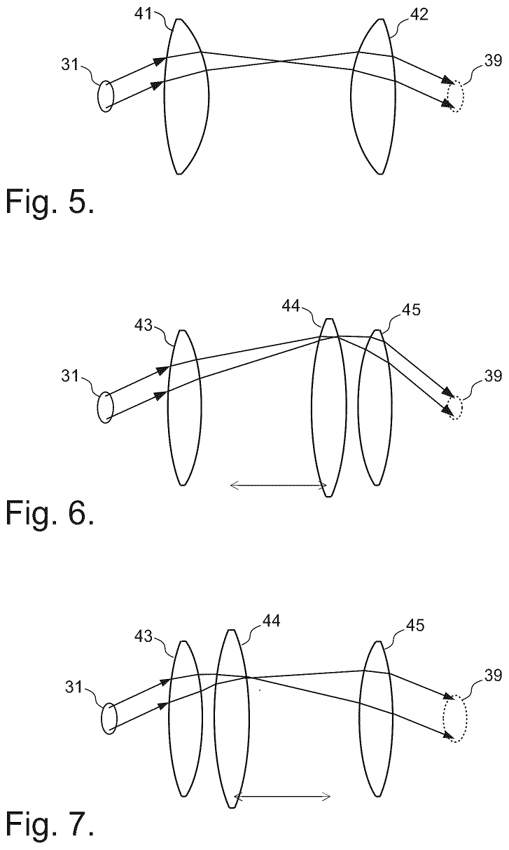

FIG. 5 shows a 4-f dual-telecentric relay system with two lenses 41, 42, whose principal planes are separated by the sum of their focal lengths. The scan mirror 31 is located at the front focal plane of lens 41, and the optical relay forms an image 39 of the scan mirror at the rear focal plane of lens 42. Similar fixed magnification configurations are widely used in other instruments, and ensure that the image of the scan mirror, remains centred in the subject pupil independent of scan beam deflection and adjustment of beam vergence for focus.

FIG. 6 shows a modified configuration with an additional lens 44 interposed between scan lens 43 and tube lens 45. The axial position of lens 44 can be varied. Close to tube lens 45, the pupil magnification is less than unity but the sine of the deflection angle is increased in inverse proportion to the magnification.

FIG. 7 shows the behaviour with lens 44 now positioned close to the scan lens 43, resulting in greater magnification of the image 39 in the subject's pupil, but with a proportionally smaller deflection angle and field of view. In general, there are two positions for lens 44 which give identical image positions but different magnification. At intermediate positions, the image is displaced closer to the relay. Depending on the magnification range, this image displacement may be acceptable. To suppress the image displacement, lens 44 is replaced by two or more lens elements whose separation can be varied, such that the power of the combination also varies. With two degrees of freedom, stable positioning of the mirror conjugate within the subject pupil is possible over a range of magnifications.

In a preferred embodiment, changing the magnification of the optical relay between the scan mirror and subject does not change the distance between scan head and subject, and maintains the location of the scanned beam tilt axis within the subject's pupil.

It will be evident to one ordinarily skilled in the art that with single element lenses, chromatic and other aberrations will be introduced by the relay system. These geometric aberrations are greatly suppressed if the individual lenses 43, 44, 45, are each replaced by lens groups comprising elements with complementary chromatic dispersions (variations of refractive index with wavelength) and surface curvatures such that the location of the beam tilt axis in the pupil is stable, the spot focused at the retina is substantially aberration-free and located in the same focus plane for the various magnifications supported and over the range of excitation and detection wavelengths of interest. Computer simulation and optimisation of detailed lens parameters to meet specific performance requirements is common practice and supported by software such as Zemax OpticStudio or Synopsys' Code V.

In an alternative embodiment, lens 44 is a negative power diverging lens group, rather than a converging lens. With comparable clearances between scan mirror and scan lens 43, and between tube lens 45 and subject, this combination can provide a similarly wide field of view at the subject with a smaller maximum diameter for the zoom element 44. A disadvantage is that higher converging powers are needed for the outer elements, 43, 45, so that for the same level of aberrations their design is potentially more complex, and the overall length of the optical relay assembly is greater than with a positive zoom group.

By way of example only, potential combinations of beam width and field of view appropriate for use with a scan mirror with 1 mm diameter and 50° diagonal field of view are:

| | Beam | Diagonal |
|---|---|---|
| Magnification | width | field of view |
| 0.75 | 0.62 mm | 68° |
| 1.0 | 0.82 mm | 50° |
| 1.3 | 1.1 mm | 38° |
| 1.7 | 1.4 mm | 33° |

For the largest scan angles at the lowest magnification, there is risk of vignetting unless the zoom group and tube lens diameters are rather large. Depending on performance requirements, it may be expedient to use smaller diameter lens elements, resulting in a smaller but more circular field of view, and sacrificing the extreme corners of the rectangular scan of the mirror. Apart from considerations of cost and weight, smaller diameter lenses result in a more compact optical relay and enhanced visibility of and access to the subject.

In a preferred implementation, a beam splitter 14 separates the incident and returned beams where both are substantially collimated. In one implementation, the collimated beam is focused onto an aperture 64 which is conjugate with the scanned spot at the retina, and light transmitted though the spatial filter aperture coupled into a multimode optical fibre, as described by Blaha. In a preferred implementation a precision fibre coupler focuses the light onto the core of a few-moded or multimode optical fibre, such that the fibre core constitutes the spatial filter.

In the absence of aberrations, the transverse resolution of a confocal SLO is improved by a factor 1.4 with a single mode input and matched single mode output such that the images of the mode fields at the retinal conjugate have identical size and location for each fibre. In practice, aberrations broaden the image spot, and a matched mode field configuration has poor light collection efficiency and degrades the signal to noise ratio. With typical aberrations an effective compromise is to select a core diameter for the collection fibre such that its image at the retina is a small multiple of the diameter of the diffraction-limited Airy disk from a uniformly illuminated pupil with the same diameter as the incident scan beam. Multiples between 1× and 5× the Airy disk diameter provide good collection efficiency while maintaining acceptable transverse and axial spatial resolution.

To maximise coupling efficiency, the numerical aperture of the detection fibre should be no smaller than the numerical aperture of the return beam focused onto the fibre by the lens 62 of the fibre coupler.

By way of example only, commercially available single mode fibres are typically specified with numerical apertures in the range 0.1 to 0.14. Fibre with cut-off wavelength 600 nm will have a core diameter of approximately 4 μm and exhibit low bending losses for excitation wavelengths up to 780 or 830 nm. As discussed above, with accurately centred source coupling or appropriate mode filtering, shorter wavelengths around 480 nm can be used. If similar focal length coupling lenses are used for both excitation 12 and detection 62, then spatial filter 64 or fibre 61 core diameters in the range 8 to 25 μm can be used. For example, telecommunications fibres with single mode cut-off 1250 nm or 1400 nm have core diameters of 9-10 μm. For imaging modalities such as auto-fluorescence the returned signal is often low, and a 50 μm core 0.2 NA multimode fibre will collect more light and offer better signal to noise performance with only a modest loss of resolution.

A potential problem with instruments of this type is Fresnel reflection at interfaces between the beam splitter and the subject, wherever there is a change in the refractive index of the propagation medium. There are well-known mitigating techniques to minimise the power from such reflections reaching the detector. Dielectric anti-reflection coatings on air-glass interfaces are recommended in any case to improve transmission. For planar and large radius surfaces, a small tilt away from normal incidence can deflect the reflected beam away from the confocal spatial filter, without introducing significant optical aberrations. Apertures or masks can block reflections from specific surfaces, but this becomes difficult when the number of surfaces is large and ray vergence can vary over a wide range as retinal focus and pupil magnification are adjusted.

Fresnel reflection preserves a linear state of polarisation, so an effective means to suppress reflections is to insert a linear polariser in the detection beam, oriented orthogonal to the polarisation of the illumination source. The output from semiconductor diode lasers is linearly polarised at source, and can be transmitted through polarisation-maintaining single mode optical fibres which preserve both the linear polarisation state and the high spatial coherence of the source.

In a preferred embodiment, illumination is provided from a linear polarisation-maintaining optical fibre via a connectorised optical fibre coupler. Rotational misalignment between laser diode and fibre pigtail, and polarisation cross-coupling in the fibre can perturb the polarisation state, limiting the degree of polarisation extinction possible by the detector polariser 63 so an additional polariser 13 aligned with the nominal polarisation axis of the input fibre is preferred.

In another preferred embodiment, additional polarisation discrimination is provided by making the beam-splitter 14 a polarizing beam-splitter.

The optical power which can be coupled into the subject's eye is constrained by the need to avoid causing retinal damage. Power at the pupil is typically less than 1 mW, especially at shorter visible and ultra-violet wavelengths where there is greater risk of photochemical injury to the retina. In imaging modalities such as autofluorescence the returned signal can be very low, requiring stacking of multiple image frames to collect a usable signal. A detector with high quantum efficiency, low noise and low dark signal is required. Photomultipliers and silicon avalanche photodiodes (Si APD) have been used. A preferred embodiment uses multi-pixel photon counter (MPPC) detectors, also known as silicon photomultipliers (SiPM), with high avalanche gain, low dark current and lower operating voltages than typical vacuum tube photomultipliers and Si APDs.

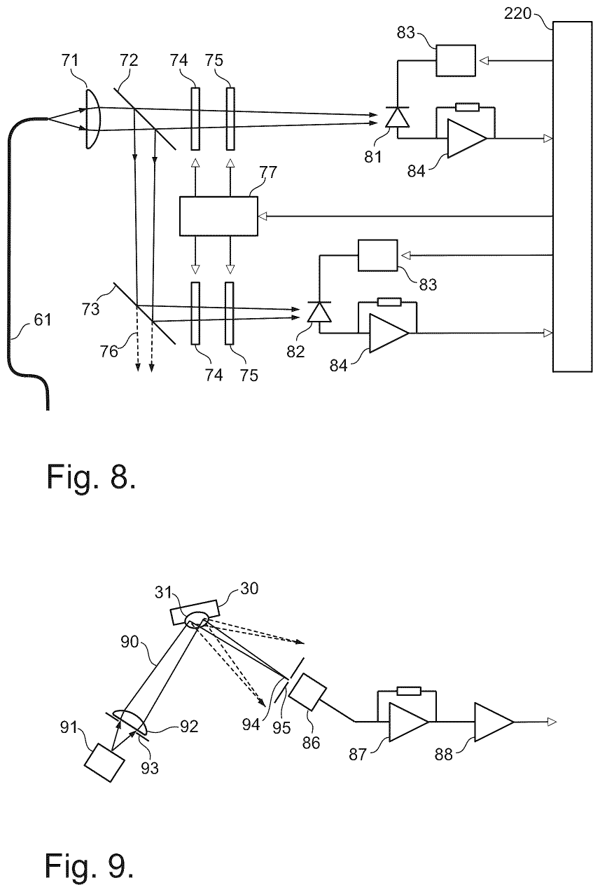

FIG. 8 shows a preferred embodiment of the detection module 70. Light guided by the flexible detector waveguide 61 is collimated by lens 71 and directed onto photo-detector 81. Bias voltage source 83 controls the avalanche gain and transimpedance amplifier 84 transforms the current output of the detector to an electrical signal with output impedance matched to the input of the signal processing module 220.

To support simultaneous imaging at multiple wavelengths a dichroic beam splitter 72 diverts one or more wavelength bands to a parallel detection path using a second detector 82 which may be chosen with a different spectral response to first detector 81. Some imaging modalities, notably auto-fluorescence, return very low light levels at the wavelengths of interest, and it is particularly important to reject the excitation wavelengths. Band-pass and edge blocking filters 74, 75 are inserted as required for each imaging modality. Preferably an electro-magnetic or other means of insertion 77 allows rapid selection of filter combinations under direct user or programmed control. It will be apparent that additional parallel detection channels can be added by replacing mirror 73 by another dichroic beam splitter, such that the transmitted light 76 is processed by one or more additional filter and detector combinations.

In a preferred embodiment at least one axis of the scan is provided by a resonant oscillation of the scan mirror. The mirror deflection varies sinusoidally with time and the signal is captured on both forward and backwards sweeps of the mirror. In principle the signal could be sampled at a variable rate such that samples are collected at times corresponding to a uniform angular spacing of points across the retina. In practice, analogue to digital converters (ADC) with adequate resolution and bandwidth typically employ a pipelined architecture such that sampling is synchronised with a constant frequency clock. In this case the stream of ADC samples must be interpolated to convert the raw data to a rectangular image raster of more uniformly spaced pixels.

It is particularly important to synchronise or otherwise align the signal samples corresponding respectively to forward and backward mirror sweeps. If electrical samples are delayed with respect to the mirror oscillation, image pixels will be shifted behind the angular motion of the mirror, so in opposite directions on alternate lines. Pham (2011) describes a capacitive sensor integrated into the mems module to detect zero crossings at the mid-point of each line. A potential problem with such on-chip capacitive sensors is noise, resulting in 86 ns rms jitter in the device reported by Pham. The underlying mechanical resonant oscillation has greater short-term stability, so timing errors can be reduced by averaging over multiple oscillation periods.

An alternative is to use an optical zero crossing detector (OZCD) to track phase drift and systematic variations in mirror resonant frequency. In a preferred implementation, shown schematically in FIG. 9 a converging beam of light 90, formed from source 91 and lens 92 is directed onto the scan mirror 31 and the reflected beam brought to a focus at an optical detector comprising a linear sensor element 86. An aperture 93 restricts the beam width near the source and minimises scattering by ensuring that the beam is confined to the scan mirror, and does not illuminate other parts of the MEMS module 30. The sensor is preferably masked by a slit 95 aligned such that at zero crossings of the fast resonant scan, the slit is in the plane swept out by the OZCD beam as the vertical deflection varies and is substantially orthogonal to the reflected beam 94 when the vertical deflection is near its mid-point.

A pre-amplifier 87 and comparator 88 convert the analogue output from sensor 86 to a binary logic-level pulse each time the OZCD beam 94 reflected from the scan mirror illuminates the slit 95. The pulse width depends on the beam width, the detector or slit width and the distance from the scan mirror, and is preferably in the range 0.05 to 1.0 μs.

The OZCD pulses are processed by control module 210. The zero crossing time corresponding to each pulse is preferably calculated as the mean of the arrival times of the rising and falling edges. Using the mean greatly reduces the sensitivity to drift in laser output power or to changes in signal level with mirror vertical deflection.

OZCD zero crossing times calculated in this way remains sensitive to the precise transverse position of the slit 95 and its angular alignment. Systematic delays can be calibrated and corrected in the software or firmware of the control system, but such corrections depend on the velocity of the scan mirror, and hence the deflection amplitude. It is desirable that deflection amplitude can be varied under user control, in concert with the variable magnification provided by the second optical relay system 40, in order to optimise signal to noise ratio and image resolution according to the imaging modality in use.

In a preferred embodiment, the need to re-calculate timing corrections for each mirror deflection amplitude is avoided by determining the timing of the centre point of each line, t_{C}, using a weighted average of three or more zero crossings for each scan line.

In general, a robust estimate of the time, t_{DC}, at which the mirror oscillation changes direction, is given by the average of two successive OZCD timings, each calculated as the mean of the rising and falling edges times for each individual pulse. For a sinusoidal oscillation, this turning point is one quarter period in advance or lagging behind the zero crossing, so an improved estimate of the true zero crossing is:

$${t}_{C} = {t}_{D C} \pm T / 4$$

Where the oscillation period, T, is equal to the time between OZCD pulses from successive lines scanned in the same direction.

More specifically, if samples from multiple lines are buffered and times of the current zero crossing, t_{ZC}(n), previous t_{ZC}(n−1) and succeeding zero crossing t_{ZC}(n+1) are saved, then an improved estimate of the n^{th} zero crossing time is:

$${t}_{C} ( n ) = 0.25 {t}_{ZC} ( n - 1 ) + 0.5 {t}_{ZC} ( n ) + 0 \text{.25} {t}_{ZC} ( n + 1 )$$

Alternatively, if the ADC clock phase is adjusted before the start of each line, then a robust estimate of the zero crossing time for line n is calculated from the three preceding zero crossing times as:

$${t}_{C} ( n ) = 1 \text{.25} {t}_{ZC} ( n - 1 ) + 0.5 {t}_{ZC} ( n - 2 ) - 0 \text{.75} {t}_{ZC} ( n - 3 )$$

A signal processing module 220 is provided which, as a minimum, comprises an analogue to digital converter which transforms the continuous electrical signal from the detector to a sequence of digital representations of the signal at each sample instance. Further transformations are required to generate an image suitable for display or subsequent processing by generic software tools.

Samples are collected on both forward and backward sweeps of the resonant mirror oscillation. The order of ADC samples from alternate lines must be reversed.

A delay between the OZCD zero crossing time and the nearest ADC sample time corresponds to a spatial displacement between lines respectively from forward and backwards sweeps of the mirror. This can be corrected by interpolating between ADC samples. Temporal alignment by interpolation is not necessary if the phase of the ADC clock is adjusted with sufficient precision before the start of each line.

ADCs with sufficiently high sample rate and resolution typically employ a pipelined architecture which requires a constant frequency sample clock. The scan mirror resonant axis deflection is a sinusoidal function of time, so that adjacent ADC samples correspond to a larger physical separation in the centre of each line than at the edges. The un-corrected image is distorted and features are stretched out towards the extremes of the (horizontal) resonant axis scan.

In addition to the sinusoidal distortion, there is distortion associated with the geometry of the mirror tilt axes and the orientation of the incident beam with respect to the centre of the field of view. The general effect is barrel distortion combined with a curvature of the image field away from the direction of the incident beam. For beam deflections at the scan mirror of +10° in both horizontal and vertical directions (20°×20° field of view at unity magnification), image distortion is typically less than 5%, but increases quadratically with deflection amplitude.

It will be appreciated by one skilled in the art that there are myriad means to embody the distortion correction processes outlined above, including but not limited to one or some combination of application-specific integrated circuit (ASIC), field programmable gate array (FPGA), graphics processing unit (GPU) or microprocessor. One approach is to implement minimal processing in the instrument itself and use the host computer 300.

In a preferred embodiment the timing alignment, sinusoidal and geometric projection transformations are applied in the signal processing module 220 and system control module 210, and corrected image frames transferred to the host computer 300 as either discrete images or as a video stream. Use of an industry standard hardware interface such as Universal Serial Bus (USB) and a well-established communication protocol such as USB Video Class (UVC) provides users the ability to integrate the instrument with existing hardware and software tools.

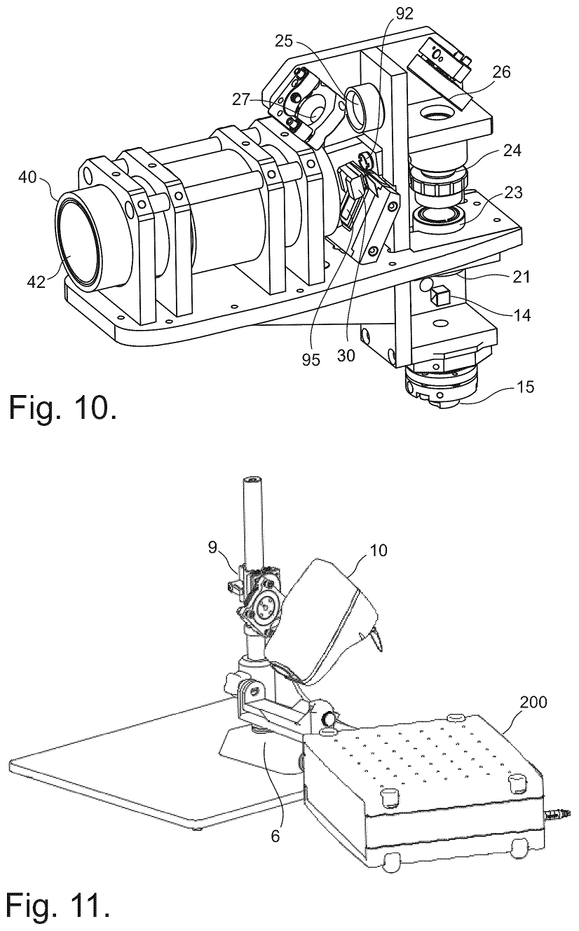

FIG. 10 shows an embodiment of the scan optics in which a relatively long optical path is achieved within a compact scan head by folding the optical path. Excitation light is delivered from a connectorised optical fibre collimator 15 and directed through beam splitter 14. Fold mirror 26, inclined at 45° to the beam is located between lenses 24 and 25 of the first optical relay, with a second 45° fold mirror 27 directing light onto the scan mirror of the MEMS module 30 via fold mirror 32 (not visible in this view). Two mirrors (not shown) direct light respectively from the OZCD collimator 92 onto the scan mirror, and from the scan mirror to the slit 95 of the OZCD detector.

FIG. 11 shows the scan head 10 with its outer shell in place. Translational and rotational adjustments 9 allow precise control of the scan head position and orientation with respect to a cradle 6 provided to support and orientate a small animal subject.

Techniques commonly employed and combined in ophthalmic imaging include reflectance imaging at one or more wavelengths, fluorescence imaging using dyes or auto-fluorescence with a range of excitation wavelengths and optical coherence tomography (OCT). If only a limited number of different sources are required, single mode fused fibre WDM (wavelength division multiplex) couplers are stable with low insertion losses. When more flexible wavelength selection is required, expanded beam couplers and dichroic filters are preferred.

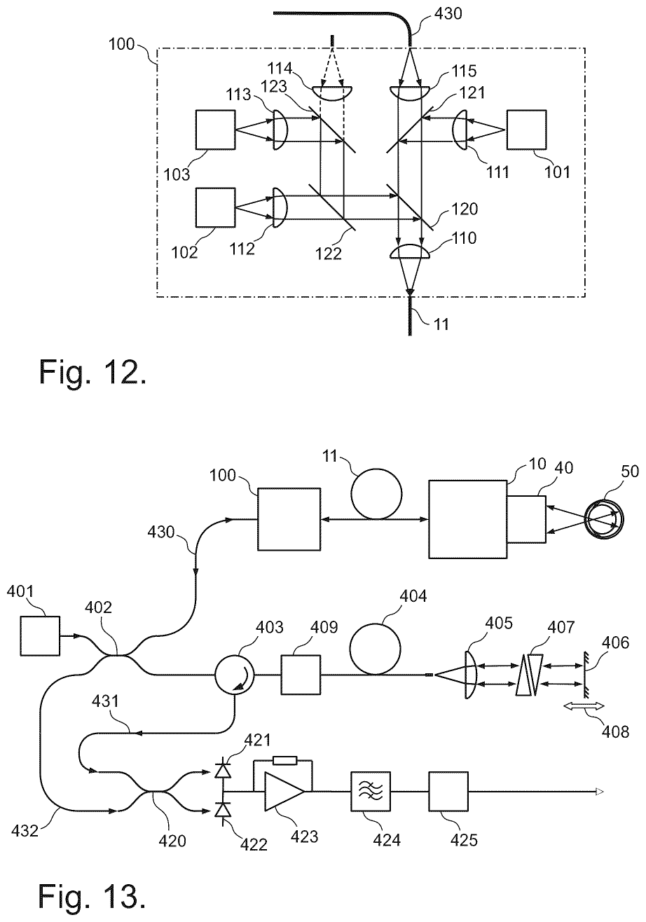

By way of example only, FIG. 12 shows a means for flexible support of multiple excitation sources. The output from laser sources 101 and 102 are collimated by lenses 111 and 112, the beams combined by dichroic filter 120 and coupled into fibre 11 by lens 110. For 780 nm and 488 nm sources, a long pass filter with cut-on wavelength between 600 and 750 nm would be appropriate. A second ultra-violet or visible source 103 could be added via collimating lens 113 and dichroic filter 122. Reflector 123 could be either a mirror, or a third dichroic filter to support a third UV or visible source, fibre-coupled via lens 114. In the near infra-red branch, dichroic filter 121 and lens 115 couple light from optical fibre 430. For optical coherence tomography, transmission is bi-directional, returning back-scattered light to the OCT module via interconnect 430.

FIG. 13 shows one embodiment of OCT. A source 401 with high spatial coherence and low temporal coherence is required. Super-luminescent diode (SLD) sources are available at a range of near infra-red wavelengths, but super-continuum laser sources are also used. The achievable axial resolution is inversely proportional to the optical bandwidth of the source, as described by Tomlins and Wang (2005). For an 850 nm source with 50 nm bandwidth (full width at half maximum), the axial resolution in air is approximately 6.4 microns, corresponding to around 4.8 microns depth in aqueous tissue.

A directional coupler 402 directs light via optical cable 430 from the source to the excitation module 100 which is coupled to the scan head 10 via optical cable 11. Optical relay 40 images the scan mirror into the eye 50 of the subject. Reflected and scattered light is transmitted back through the system, returning to the directional coupler 402. A second output port of coupler 402 directs light to the reference path comprising optical circulator 403, phase modulator 409, collimator lens 405 and mirror 406. The optical fibre cable 404 is selected to match the optical path length through the signal cables 430, 11, the scan optics and transmission to the subject's fundus 52. Adjustment to the reference path length is provided by translation mechanism 408.

Preferably the optical fibre interconnect 11, and the internal OCT interconnects including 404, 430, 431, 432, are polarisation maintaining and single mode, supporting orthogonal linearly polarised modes over the wavelength range emitted by the source 401.

Optical circulator 403 directs back-reflected light from the reference path through fibre 431 to 50/50 directional coupler 420 which preferably distributes power equally from either of its two input ports to the two output ports. Signal light returned from the subject is coupled via fibre 432 to the second input port. The two output ports emit respectively the sum and the difference of the signal and reference optical input fields. The photo-current in photodiodes 421, 422 is predominantly a common mode signal proportional to the sum of the signal and reference optical powers. Superimposed is a differential signal proportional to the square root of the product of signal and reference powers. The photodiodes are connected in series in a balanced detector configuration, such that the common mode photo-currents cancel. The differential beat currents combine in phase at the input to the transimpedance amplifier 423.

The magnitude of the beat signal depends not only on the amplitudes of the back-scattered and reference optical signals, but also on their respective instantaneous optical phases. The phase modulator 409 is driven to ensure that the optical delay in the reference varies by at least one wavelength or through more than 360 degrees of phase to produce a varying beat signal whose amplitude can be measured. Bandpass filter 424 selects frequencies centred on the modulation frequency, or as discussed in U.S. Pat. No. 5,975,697 by Podoleanu and Jackson, a harmonic of the modulation frequency. The amplitude of the alternating beat frequency is extracted by rectification or other means in demodulator 425, digitised, and transmitted to signal processing and control modules 220, 210.

The beat signal amplitude is highest when the optical group delay is identical in signal and reference arms at all optical frequencies. Beat amplitude and axial resolution are both degraded if chromatic dispersion causes wavelength-dependent group delay differences between the two paths. A first order dispersion correction is by an opposing pair of triangular cross-section glass prisms 407, which allows the effective thickness of dispersive material in the reference path to be varied without significant change to the alignment of the expanded beam. For very broad spectral bandwidths, correction of higher order dispersive terms is possible using two pairs of compensating prisms, each pair using glass with different dispersive power. Kowalevicz et al. (2002) use a first compensator made from BK7 crown glass and a second compensator using a higher dispersion flint glass.

Optical circulator 403 ensures that light is directed though optical fibre 431 and is not propagated back through directional coupler 402 into source 401. Such back-reflections can cause optical instability, in extreme cases resulting in laser oscillation and potentially optical damage. An alternative is to insert an optical isolator between source 401 and coupler 402.

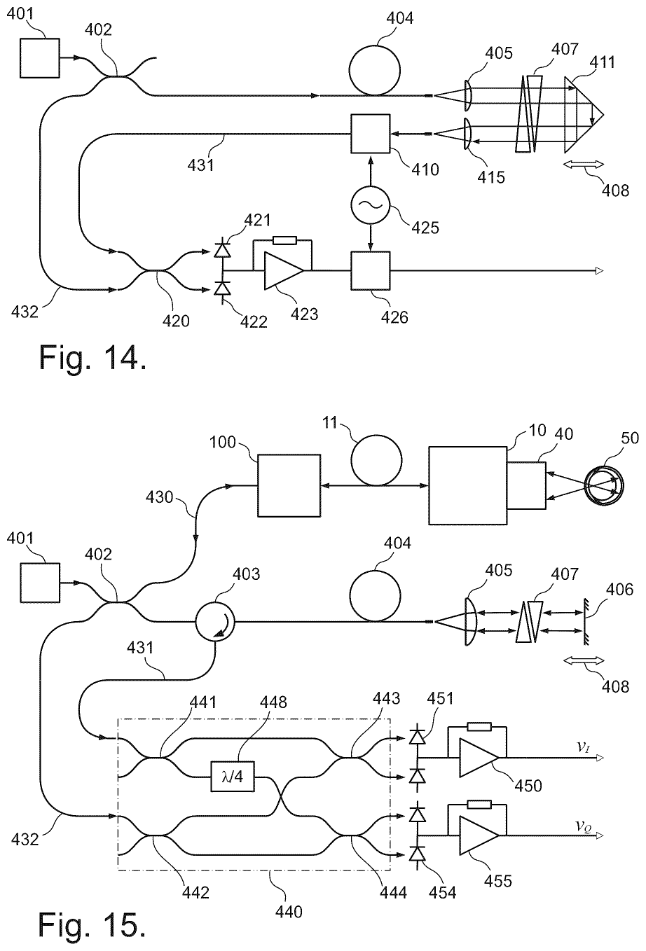

FIG. 14 shows an alternative reference path and detection scheme. The mirror is replaced by a retro-reflector mirror or prism 411, and the laterally displaced reflected beam coupled into a single mode optical fibre by lens 415 and transmitted to acousto-optic modulator 410. The frequency-shifted reference signal is transmitted by fibre 431 to 50/50 directional coupler 420, and mixed with the back-scattered signal from fibre 432. Coherent beating between reference and signal is measured using balanced detection by photodiodes 421 and 422, and the difference photocurrent amplified in transimpedance amplifier 423. The beat signal is synchronous with the frequency shift induced by the acousto-optic modulator (AOM). Phase sensitive or coherent detection in electrical mixer 426 uses the AOM radio frequency (RF) drive signal 425 as local oscillator to produce a signal proportional to the photodiode beat signal. Preferably quadrature local oscillator waveforms are generated and outputs from in-phase and quadrature RF mixers are combined so that the output signal is proportional to the RMS amplitude of the beat signal, independent of the optical and local oscillator phases.

FIG. 15 shows an embodiment which avoids the need for high frequency phase or acousto-optic modulation using a detection technique common in high-speed optical communication systems. The signal and reference paths are similar to those described in FIG. 13, with the absence of a phase modulator. The reference fibre 431 and signal fibre 432 are coupled to the input of 90-degree hybrid 440. For clarity, an optical waveguide embodiment of the hybrid is illustrated. Each input is split equally by directional couplers 441, 442, and copies of both signal and reference applied to directional couplers 443, 444. The optical path lengths of the internal interconnects are carefully matched except for one of the reference cross-connects in which an incremental delay of one quarter of the period of the centre wavelength of the source is added 448. Balanced detectors, 451, 454, at the outputs of the couplers 443, 444, deliver differential photocurrent inputs to transimpedance amplifiers 450, 455 whose outputs are proportional to the in-phase and quadrature components of the beat signal. The reflectance amplitude is proportional to the root sum of squares of the two transimpedance outputs:

$${v}_{m} = \sqrt{{v}_{I}^{2} + {v}_{Q}^{2}}$$

While planar waveguide implementations of 90-degree hybrids are possible it can be difficult to maintain an accurate quadrature relationship without temperature stabilisation. Bulk optic implementations, such as the Michelson interferometer-based design offered by Optoplex Corporation, provide equivalent functionality and are a stable and robust alternative.

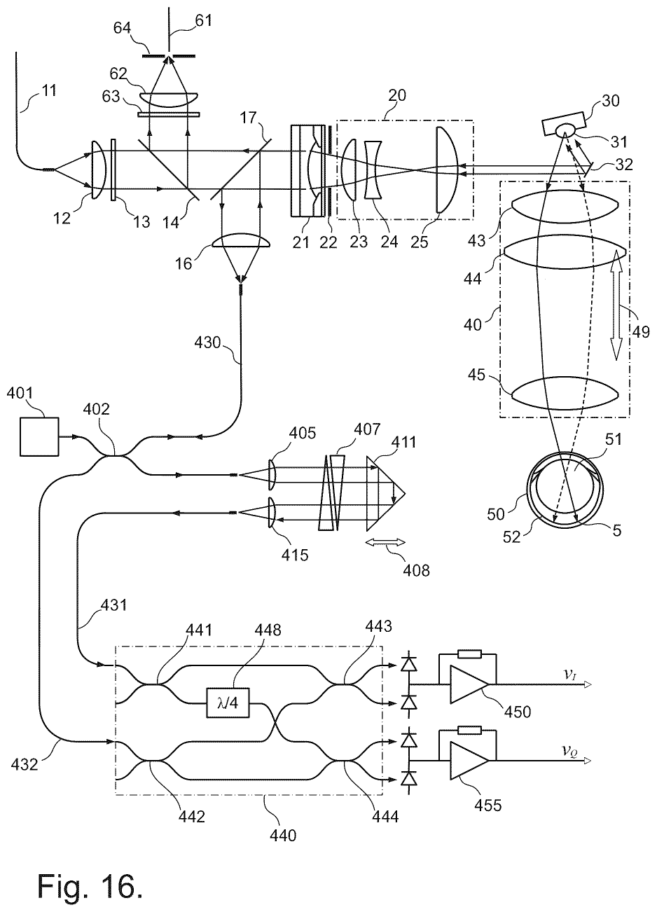

Coupling the OCT probe and return signals through the source multiplexer 100 offers convenience and flexibility. However, multiple fibre to free space coupling losses, the insertion losses of dichroic filters 110, 115, and scan head beamsplitter 14 degrade the sensitivity and signal to noise ratio. FIG. 16 shows a preferred implementation in which OCT illumination is coupled via a collimator 16 and dichroic filter 17, located between beamsplitter 14 and focusing lens 21. Filter 17 selectively reflects light in the wavelength range of the OCT source, and transmits other, typically shorter, imaging wavelengths. The same configuration can be used with the frequency or phase modulator implementations shown in FIGS. 13 and 14.

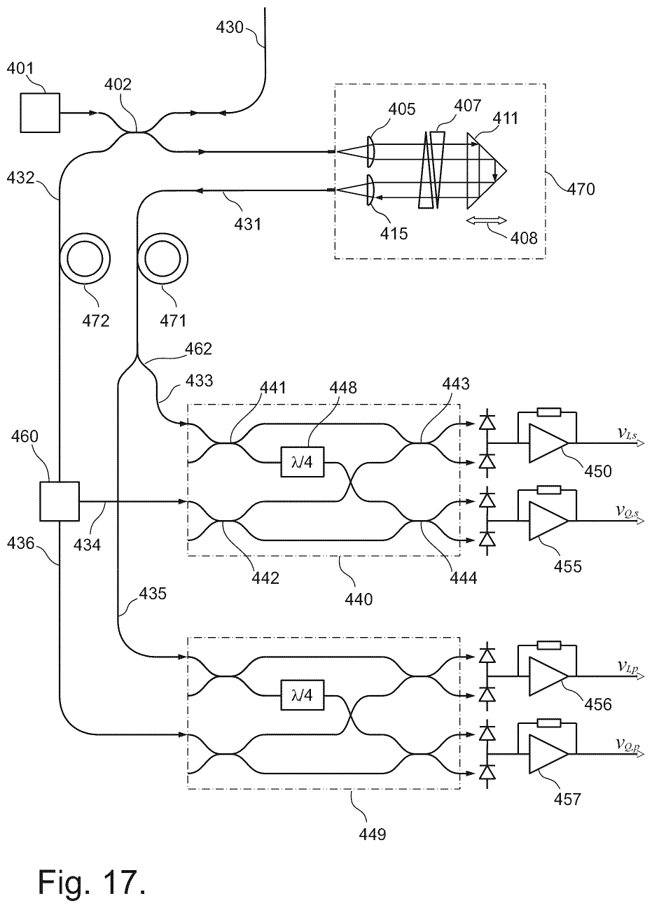

FIG. 17 shows a polarization diversity OCT implementation. Light returned from the subject is transmitted by polarization-maintaining fibres 430, 432 to polarization demultiplexer 460 and split into two orthogonal polarization states, transmitted by fibre 434 to 90 degree hybrid 440, and by fibre 436 to a second hybrid 449. The reference signal from delay module 470 is split by 3 dB coupler 462, with outputs 433, 435, coupled to the reference inputs of the two optical hybrids. The four balanced detector outputs from transimpedance amplifiers 450, 455, 456, 457 are preferably sampled simultaneously by analog to digital converters. Polarisation fading is suppressed by computing a reflectance amplitude proportional to the root sum of squares of the four outputs:

$${v}_{m} = \sqrt{{v}_{I , s}^{2} + {v}_{Q , s}^{2} + {v}_{I , p}^{2} + {v}_{Q , p}^{2}}$$

The in-phase and quadrature field amplitudes in two orthogonal polarization states describes the rotation of the polarization state of the signal with respect to the reference at the detector, and is a basis for polarization OCT imaging.

For some en-face OCT applications, the translation mechanism 408 for reflector 411 in optical delay module 470 may respond too slowly. Variable delay modules comprising an optical fibre, with a piezo electric means to stretch the fibre provide a more rapid response. Degradation of axial resolution due to unbalanced fibre chromatic dispersion in signal and reference paths is minimized using matched delay modules 472, 471, in signal path 432 and reference path 431, driven partly in anti-phase to minimize differences in net dispersion at the interferometers.

### REFERENCES

U.S. Pat. No. 3,016,000, 1962, Y Sato and K Noyori, “Portable oculi fundus camera”
U.S. Pat. No. 4,213,678, 1980, O. Pomerantzeff and R. H. Webb, “Scanning laser ophthalmoscope for examining the fundus of the eye”.
U.S. Pat. No. 5,071,246, 1991, E. Blaha, G. Gaida, Confocal Scanning Ophthalmoscope.
U.S. Pat. No. 5,975,697, 1999, A. G. Podoleanu and D. A. Jackson, “Optical mapping apparatus with adjustable depth resolution”.
U.S. Pat. No. 7,993,000 B2, 2011, N. A. Massie, “Method and apparatus for imaging an eye of a small animal”.
U.S. Pat. No. 9,743,831 B2, 2017, D. Gray, D. Swan, M. Thomson, “Retinal imaging apparatus and method”.

US 2010/0020379 A1, 2010, Yoram Lubianiker, “Gimbaled scanning micro-mirror actuation scheme and architecture”.
U.S. Provisional Patent Application 63/380,605, 24 Oct. 2022, Peter West and Alan Robinson, “Scanning Laser Ophthalmoscope for Small Animals”
S. G. Adie et. al, “Audio frequency in-vivo optical coherence elastography”, Phys. Med. Biol. Vol. 54 (2009) 3129-3139.
Johannes F. de Boer, Christoph K. Hitzenberger & Yosihiaki Yasuno, “Polarization sensitive optical coherence tomography-a review”, Biomedical Optics Express 1838, vol. 8, no. 3, March 2017
Lee R. Ferguson, James M. Dominguez II, Sankarathi Balaiya, Sandeep Grover, Kakarla V. Chalam, “Retinal Thickness Normative Data in Wild-Type Mice Using Customized Miniature SD-OCT”, PLOS, Jun. 27, 2013, DOI: 10.1371/journal.pone.0067265.
Ying Geng et. al., “Optical properties of the mouse eye”, vol. 2, no. 4, pp 717-738, Biomedical Optics Express, April 2011.
A. M. Kowalevicz et. al. “Ultrahigh resolution optical coherencetomography using a superluminescent lightsource”, Optics Express, vol. 10, (2002), p 349.
D. D Pham et. al. “Position Sensing and Electrostatic Actuation Circuits for 2-D Scanning MEMS Micromirror”, Defense Science Research Conference and Expo (DSR), 3-5 Aug. 2011, DOI: 10.1109/DSR17494.2011.
S. Remtulla & P. E. Hallett, “A schematic eye for the mouse and comparisons with the rat”, Vision Rs. Vol. 1, pp 21-31, 1985.
Warren J. Smith, “Modern Optical Engineering”, 3rd ed., McGraw-Hill, 2000.
A. W. Snyder and J. D. Love, “Optical Waveguide Theory”, ISBN 0-412-09950-0.
P. H. Tomlins and R. K. Wang, “Theory, developments and applications of optical [0157] coherence tomography”, J. Phys. D: Appl. Phys. 38 (2005) 2519-2535.
R. H. Webb and G. W Hughes, “Scanning Laser Ophthalmoscope”, IEEE Transactions on biomedical engineering, BME-28, no. 7, July 1981, pp 488-492.
R. H. Webb, G. W. Hughes and F. C. Delori, “Confocal scanning laser ophthalmoscope”, Applied Optics, vol. 26, no. 8, 15 Apr. 1987, pp 1492-1499.
D. L. Yan et. al., “The phase locked loop for MEMS horizontal scanning control of micro-laser projection ASIC”, International Symposium on Integrated Circuits, 2011.
Pengfei Zhang et. al, “Effect of scanning beam size on the lateral resolution of mouse retinal imaging with SLO”, Optics Letters, vol. 40, no. 24, Dec. 15, 2015.
Xiangtian Zhou et. al, “The Development of the Refractive Status and Ocular Growth in C57BL/6 Mice”, Investigative Ophthalmology & Visual Science, December 2008, Vol. 49, No. 12.

1. An ophthalmic imaging apparatus comprising an optical coherence tomography device, the optical coherence tomography device comprising:
an optical source with low temporal coherence;
an optical splitter to direct the output from said optical source simultaneously to a subject and to a reference optical path comprising a delay module to vary the optical path length of said reference path optical path; and
an optical combiner to combine light reflected or scattered from said subject with light transmitted through the reference optical path,
wherein in-phase and quadrature interference beat signals are generated by a quadrature detector comprising a 90-degree optical hybrid and balanced optical detection.

2. A method of imaging the fundus of a small animal, comprising using the ophthalmic imaging apparatus according to claim 1.

3. An instrument comprising an optical scan head, said scan head comprising:
an optical excitation light source with high spatial coherence;
a first optical system to direct light from said optical excitation source onto a scan mirror;
a variable focusing optic to vary the vergence of the excitation light directed by said first optical system onto said scan mirror;
a second optical system which forms an image of the scan mirror near the pupil of the eye of a subject such that the excitation light is brought to a focus in or near the retina of the subject's eye;
a beam splitter to separate light returned from said retinal focus point from the path of the excitation beam after said returned light is reflected by the scan mirror, and a lens to focus it to a point conjugate with the retina;
a spatial filter located at said retinal conjugate;
a flexible coupler to transfer light transmitted by said spatial filter to a detection system;
wherein said scan mirror is part of a micro electro-mechanical system comprising:
said scan mirror able to tilt about a first axis substantially aligned with a first diameter of said mirror; and
a driver to excite a resonant oscillation of said mirror about said first axis and an actuator to tilt said mirror about a second axis substantially aligned with a second diameter of the mirror and orthogonal to the first axis.

4. The instrument according to claim 3 wherein the oscillation frequency about said first axis is greater than 5000 Hz and the diameter of said scan mirror is less than 2 mm.

5. The instrument according to claim 4 in which the variable focusing optic has a response time less than 30 ms.

6. The instrument according to claim 5 in which the variable focusing optic is an electro-wetting lens.

7. The instrument according to claim 3 wherein said second optical system comprises a zoom magnification actuator to vary the magnification of the image of the scan mirror formed near the pupil of the subject's eye such that the distance from said image to said second optical system is substantially constant as the magnification is varied.

8. The instrument according to claim 5 wherein the excitation source is a single mode or few-moded optical fibre whose remote end is coupled to one or more optical sources.

9. The instrument according to claim 8 wherein the excitation source optical fibre is single mode at the longest wavelength supported.

10. The instrument according to claim 9 wherein the excitation source optical fibre has normalised frequency (V-number) smaller than 3.9 at the shortest excitation wavelength.

11. The instrument according to claim 3 wherein the output from the detection system is processed by an analogue to digital converter which samples the detected signal at regular intervals whereby the times of said samples are synchronised to the resonant oscillation of the scan mirror by an optical zero crossing detector comprising:
an optical source which projects a beam of light onto the scan mirror;
an optical detector sensitive to the light from said source after reflection from said scan mirror; and
an electronic circuit to generate a synchronisation pulse from the output of said optical detector each time the angular deflection of the resonant mirror is mid-way between the two extremes of its angular deflection range.

12. The instrument according to claim 11 wherein the synchronisation time for each line is calculated from timings of the rising and falling edges of said optical zero crossing detector pulses from at least three successive scan lines such that the calculated zero crossing time is insensitive to small displacements of said zero crossing detector from the exact mid-point of said angular deflection range.

13. A method of imaging the fundus of a small animal using an instrument comprising an optical scan head in which the diameter of the optical beam projected into the eye of the subject is between 30% and 90% of the diameter of the subject's pupil; wherein the scan head comprises:
an optical excitation light source with high spatial coherence;
a first optical system to direct light from said optical excitation source onto a scan mirror;
a variable focusing optic to vary the vergence of the excitation light directed by said first optical system onto said scan mirror;
a second optical system which forms an image of the scan mirror near the pupil of the eye of a subject such that the excitation light is brought to a focus in or near the retina of the subject's eye;
a beam splitter to separate light returned from said retinal focus point from the path of the excitation beam after said returned light is reflected by the scan mirror, and a lens to focus the returned light to a point conjugate with the retina;
a spatial filter located at said retinal conjugate;
a flexible coupler to transfer light transmitted by said spatial filter to a detection system;
wherein said scan mirror is part of a micro electro-mechanical system comprising:
said scan mirror able to tilt about a first axis substantially aligned with a first diameter of said mirror; and
a driver to excite a resonant oscillation of said mirror about said first axis; and
an actuator to tilt said mirror about a second axis substantially aligned with a second diameter of the mirror and orthogonal to the first axis;
wherein the oscillation frequency about said first axis is greater than 5000 Hz and the diameter of said scan mirror is less than 2 mm; and
wherein the variable focusing optic has a response time less than 30 ms.

14. A method of imaging the fundus of a small animal using an instrument comprising an optical scan head in which the diameter of the optical beam projected into the eye of the subject is between 30% and 90% of the diameter of the subject's pupil; wherein the scan head comprises:
an optical excitation light source with high spatial coherence;
a first optical system to direct light from said optical excitation source onto a scan mirror;
a variable focusing optic to vary the vergence of the excitation light directed by said first optical system onto said scan mirror;
a second optical system which forms an image of the scan mirror near the pupil of the eye of a subject such that the excitation light is brought to a focus in or near the retina of the subject's eye;
a beam splitter to separate light returned from said retinal focus point from the path of the excitation beam after said returned light is reflected by the scan mirror, and a lens to focus the returned light to a point conjugate with the retina;
a spatial filter located at said retinal conjugate;
a flexible coupler to transfer light transmitted by said spatial filter to a detection system;
wherein said scan mirror is part of a micro electro-mechanical system comprising:
said scan mirror able to tilt about a first axis substantially aligned with a first diameter of said mirror;
a driver to excite a resonant oscillation of said mirror about said first axis; and
an actuator to tilt said mirror about a second axis substantially aligned with a second diameter of the mirror and orthogonal to the first axis;
wherein said second optical system comprises a zoom magnification actuator to vary the magnification of the image of the scan mirror formed near the pupil of the subject's eye such that the distance from said image to said second optical system is substantially constant as the magnification is varied.

15. The instrument according to claim 3 wherein the optical excitation light source comprises an optical coherence tomography device comprising:
an optical source with low temporal coherence;
an optical splitter to direct light from said optical source simultaneously through the scan head to the subject and to a reference optical path comprising a delay module to vary the optical path length of said reference optical path;
an optical combiner to combine signal light reflected or scattered from the subject and returned through the scan head with light transmitted through the reference optical path; and
a coherent interference detector to detect coherent interference between said signal light reflected from the subject and light from the reference path.

16. The instrument according to claim 15 wherein the coherent interference detector comprises a plurality of differential optical detectors wherein the phase delays between reference path and signal path differ by non-integer multiples of the optical period of the center wavelength of the low temporal coherence optical source.

17. The instrument according to claim 16 wherein the coherent interference detector comprises a quadrature detector, said quadrature detector comprising a 90-degree optical hybrid and two differential optical detectors.

18. The instrument according to claim 15 wherein the coherent interference detector comprises:
a modulator to modulate the optical phase of light in one of the reference path or the signal path;
a detection arrangement to detect the amplitude of fluctuations in said coherent interference arising from said modulation of the optical phase of light in one of said optical paths.

19. The instrument according to claim 18 wherein the modulator is an acousto-optic modulator.
